(19) United States
(12) Patent Application Publication
(10) Pub. No.: US 2026/0027266 A1
(43) Pub. Date: Jan. 29, 2026

(54) APPARATUS AND METHODS OF AN ANTIMICROBIAL COATED PRODUCT

(71) Applicant: Riverpoint Medical, LLC, Portland, OR (US)

(72) Inventors: Douglas Rowley, Portland, OR (US); Edwin Anderson, Ridgefield, WA (US)

(21) Appl. No.: 19/271,381

(22) Filed: Jul. 16, 2025

Related U.S. Application Data
(60) Provisional application No. 63/674,877, filed on Jul. 24, 2024.

(51) Int. Cl. A61L 27/54; A61L 27/10; D06M 11/79; D06M 15/564; D06M 16/00; D06M 101/20; D06M 101/32

(52) U.S. Cl. CPC A61L 27/54; A61L 27/10; D06M 11/79; D06M 15/564; D06M 16/00; A61L 2300/406; D06M 2101/20; D06M 2101/32

(57) ABSTRACT

The disclosed inventions related to an improved bioceramic coated product comprising a blended textile, a first coating layer and a second coating layer coating with a bioactive ceramic that reliably provides an optimized or enhanced antimicrobial response. The twisted configuration and bioactive ceramic properties can be tailored to manufacture an improved coated product that consistently provides an optimized antimicrobial response.

### CROSS-REFERENCE TO RELATED APPLICATIONS

This application claims the benefit of U.S. Provisional Application No. 63/674,877 entitled “Apparatus and Methods of an Antimicrobial Coated Product” filed on Jul. 24, 2024, the disclosure of which is incorporated by reference herein in its entirety.

### TECHNICAL FIELD

The disclosed inventions relate to improved systems, apparatus and methods for wound management. More specifically, the disclosed inventions relate to improved systems, apparatus and methods for antimicrobial wound management using bioactive glass for localized bioburden control.

### BACKGROUND OF THE INVENTION

Wound management represents a substantial clinical challenge due to the growing incidence of chronic skin wounds resulting from a variety of conditions, injuries and surgical wounds. The risk of infection is a key impediment to healing and a driver of increased morbidity and mortality. Infections may lead to delayed or impaired healing, and in severe cases, sepsis and death.

Therefore, efforts have been made to functionalize textiles to prevent or minimize bacterial colonization and consequent infection. Common antimicrobial textiles, such as sutures, containing antiseptic or antimicrobial molecules have shown inadequate efficacy and may induce bacterial resistance. Furthermore, other issues arise, including reduced homogeneity of the chosen antimicrobial agent or molecule and integrity of the coating or agent used on the textile.

### BRIEF SUMMARY OF THE INVENTION

In one embodiment, the method of manufacturing an antimicrobial bioceramic coated textile 5, 35, 40 comprises the steps of: twisting a first yarn to a first twist direction, a first twist angle and a first twist level and a second yarn to a second twist direction, a second twist angle and a second twist level, the first yarn including a first material and the second yarn including a second material, the first material comprising a different material than the second material; interlacing the first twisted yarn with the second twisted yarn to form a blended textile having a blended weave construction; applying a first coating layer to a portion of the blended textile, the first coating layer comprising a non-biodegradable polymer; applying a second coating layer to a portion of the first coating layer, the second coating layer comprises one or more bioceramic materials. The blended textile comprises a suture or an anchor sleeve. The twist level comprises a low or moderate twist level. The non-biodegradable polymer comprises Bionate. The one or more bioceramic particles may comprise hydroxyapatite (HA), Bioglass and/or Calcium Phosphate (CaP). The first twisted yarn material comprises greater than 50% of the blended textile. The first yarn material comprises polyester or PET and the second material comprises UHWMPE. The applying a first coating layer 25 to a portion of the blended textile comprises applying the first coating layer 25 to a portion of the first yarn 15 or a portion of the second yarn 20. The applying a first coating layer to a portion of the blended textile comprises applying the first coating layer to a portion of the first yarn or a portion of the second yarn.

In another embodiment, an antimicrobial blended textile product comprises: a textile, the textile comprising a first yarn having a first material, and a second yarn having a second material, the first yarn including a first plurality of fibers, a first yarn twist level, a first twist angle and a first twist direction and the second yarn including a second plurality of fibers, a second yarn twist level, a second twist angle, and a second twist direction, the first material is different than the second material; a first coating, the first coating disposed onto a portion of the textile, the first coating comprising a first coating material and a first coating outer surface, the first coating material is a non-biodegradable polymer; and a second coating, the second coating disposed over a portion of the first coating outer surface, the second coating comprising a second coating material, the second coating material includes one or more bioceramic materials.

In another embodiment, an antimicrobial blended textile product comprises: a textile sheath or sleeve comprising a sheath weave construction, a first yarn having a first material, the first yarn including a first twist level, a first twist angle, and a first twist direction; a core comprising a second material, the textile sheath being surrounded or disposed around at least a portion of the core, the first material is different than the second material; a first coating, the first coating disposed onto a portion of the textile sheath, the first coating comprising a first coating material and a first coating outer surface, the first coating material is a non-biodegradable polymer; and a second coating, the second coating disposed onto a portion of the first coating, the second coating comprising a second coating material, the second coating material includes one or more bioceramic materials. The core may comprise a second yarn, the second yarn may include a second twist level, a second twist angle and a second twist direction. The core may further comprise a monofilament.

### BRIEF DESCRIPTION OF THE SEVERAL VIEWS OF THE DRAWINGS

FIG. 1 depicts an front view of a first embodiment of an antimicrobial blended textile construct;

FIG. 2 depicts a front view of second embodiment of an antimicrobial blended textile construct;

FIG. 3 depicts an isometric view of a third embodiment of an antimicrobial blended textile construct;

FIGS. 4A-4B depict low and high twist configurations of a yarn;

FIGS. 5A-5B depict an alternate embodiment of twisted yarns having twisted fibers;

FIGS. 6A-6B depict magnified views of first and second coating layer onto a portion of a blended textile construct;

FIGS. 7A-7C depict front views of twisted levels, twist angles and twist directions;

FIGS. 8A-8B depict illustrations of the Test for the Determination of Minimum Bactericidal Concentration;

FIGS. 9A-9C depict illustrations of the test samples and the Graph for the Determination of the Minimum Bioactive Calcium Component of Bioglass; and

FIGS. 10A-10B depicts illustrations for the test for the Determination of Antimicrobial Efficacy via FDA recognized test.

### DETAILED DESCRIPTION OF THE INVENTION

There exists a need in industry for an antimicrobial coated textile that can be manufactured to consistently and repeatably provide an improved, enhanced or optimized antimicrobial response or properties (e.g., healing response), while preserving the mechanical properties needed to function in its intended medical application or have successful clinical outcomes. It is an object of this disclosure to provide (1) a blended textile product that includes bioactive glass; (2) a method of assembling a blended, bioactive glass coated textile product; and (3) a method of testing efficacy of the blended, bioactive glass coated textile product.

A bioactive material can be defined as a material that prompts a specific biological response between the material and the tissue that leads to the development of a bond between them and/or induce a response within the biological system. Bioactive glasses (BGs) may be used to elicit angiogenic, osteogenic, anti-inflammatory and/or antimicrobial responses. The properties or desired biological response of the BGs can be altered on the basis of the application by varying its composition, the amount of coating on the substrate and/or the surface properties of the substrate.

Currently, BGs are being used for optimizing osteointegration because they produce scaffolds for bone regeneration. BGs can be used for efficient bone tissue engineering applications as they can enhance revascularization, osteoblast adhesion, enzyme activity, and differentiation of mesenchymal stem cells. However, BGs have been shown to also have enhanced the wound healing processes due to their ability to release therapeutic ions that can stimulate various processes such as anti-inflammatory, antibacterial efficacy, and angiogenesis. In vitro and in vivo experiments have demonstrated that the ionic release of BGs causes anti-inflammatory and angiogenic growth factor expression with the subsequent increase of extracellular matrix protein deposition and the formation of blood vessel networks to provide subsequent acceleration of wound closure, as well as reduce or eliminate infections. Once bioactive glass is immersed in or exposed to physiological fluids, the bioactive glass leaches a network of ions (e.g., Na+, K+, Ca+ with H+) from the granules' surface resulting in the increase of osmotic pressure and pH, and the reduction the microbials' biofilm production. The releasing of the ions, makes the surrounding environment hostile to microbial growth without affecting the host bone or tissue. The bioactive glass antimicrobial activity is effective against a wide selection of aerobic and anaerobic bacteria, either in planktonic or sessile forms, and/or antibiotic resistant microbes. The BGs amount, the BGs adherence to the surface and/or its chemical composition can be tailored to provoke cellular activities, such as its antibacterial effect.

A biocompatible material or compound herein means that any material or substance is biologically compatible by not producing a toxic, injurious, or immunologic response in living tissue. Accordingly, a biocompatible material or substance is a term to describe that the material or substance can exist in harmony with the body. However, bioactivity is the ability of a material to elicit a specific localized biological response at the interface of the material and cells, body fluid or tissue, due to its reactive surface.

Although the following description is generally related to and illustrated with various textiles, it may include sutures, flexible tissue anchors, bandages, wound care dressings, meshes or scaffolds, and/or grafts used in wound healing to connect soft tissue. It will be understood that the methods and articles disclosed herein can also be applicable to other textile-based products and related medical applications wherein improved bioactivity or biocompatible responses or properties (e.g., healing response) are desired. Non-limiting examples of soft tissue include tendons, ligaments, fascia, skin, fibrous tissues, synovial membranes, fat, muscles, nerves, and blood vessels.

In one embodiment, the antimicrobial bioceramic coated textile product comprises a medical implant coated with at least one bioceramic material to facilitate an antimicrobial effect. More specifically, the antimicrobial bioceramic coated textile contains sufficient bioceramic material to imitate the physical structure of skin's extracellular matrix and release biologically active ions, e.g., regenerative, pro-angiogenic and anti-bacterial ions that can provoke cellular activities to regenerate the lost skin tissue and to induce new vessel formation, while keeping an anti-microbial environment.

In another embodiment, the antimicrobial bioceramic coated textile product comprises a blended or composite textile product comprising yarn or fiber twisting to facilitate increased deposition and density of the bioceramic coating on at least a portion of the antimicrobial coated textile surface to enhance antimicrobial activity. The blended or composite textile may comprise increased surface area and texturing by twisting the yarn and/or the fibers of the yarn, by braiding the twisted yarns or fibers, the plying the yarns or fibers to increase surface area, the twisting of yarns or fibers to increase wicking property, and/or any combination thereof.

In another embodiment, the antimicrobial bioceramic coated textile product comprises a blended or composite textile that may further maintain or increase various mechanical properties. Such mechanical properties may include increased density, effect on textile elongation at break, effect on textile elasticity, and/or effect on textile flexibility.

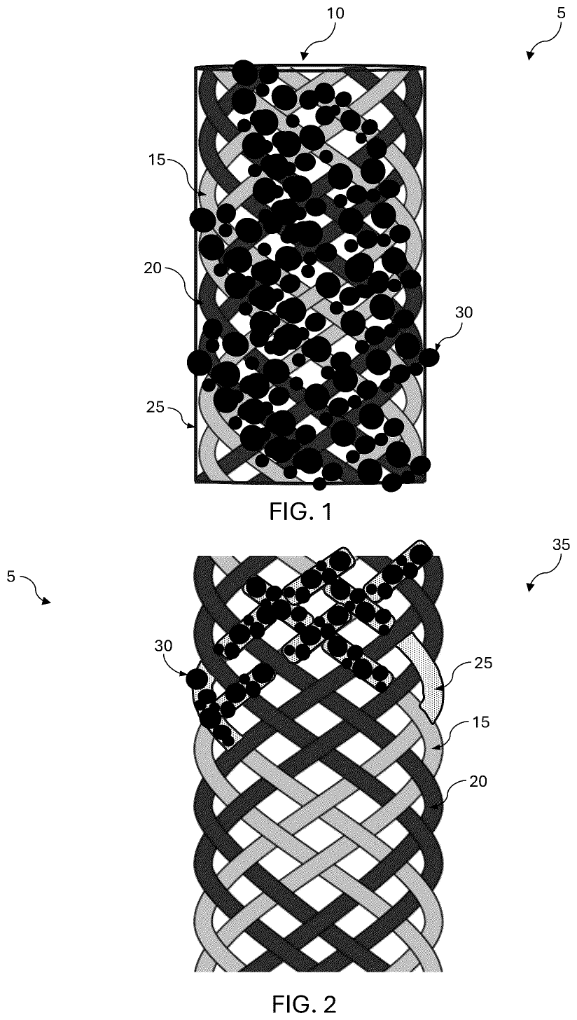

FIGS. 1-2 depict front and perspective views of a first and second embodiments of an antimicrobial bioceramic coated textile 5, 35, 40. In one exemplary embodiment, the antimicrobial bioceramic coated textile 5, 35, 40 comprises a blended textile 10, the blended textile 10 comprises a first yarn 15 having a first material, a second yarn 20 having a second material, a first coating layer 25, and a second coating layer 30. The first coating layer 25 comprises a non-biodegradable polymer, the first coating layer 25 may be deposited or adhered to a portion of the blended textile 5, 35, 40. The first coating layer 25 may be deposited or adhered to a portion of the first yarn 15 and/or the second yarn 20. The second coating layer 30 comprises one or more bioceramic materials 30. The second coating layer 30 may be deposited or adhered to a portion of the first coating layer 25. The second coating layer 30 may be deposited or adhered to a portion of the first coating layer 25 on the first yarn 15 and/or the second yarn 20. The blended textile 10 may comprise an interconnected first yarn 15 and a second yarn 20 to create a weave configuration or structure.

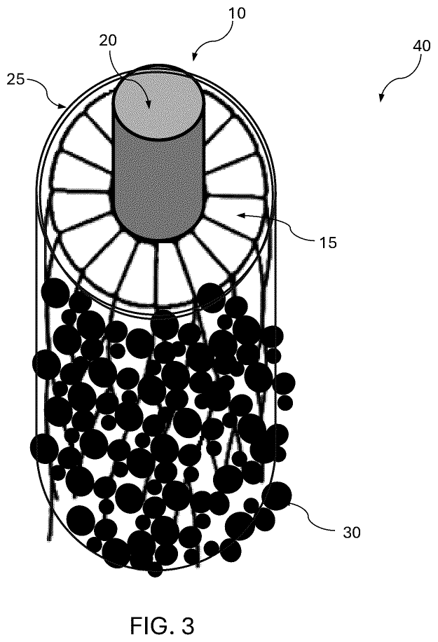

FIG. 3 depicts a third embodiment of the antimicrobial bioceramic coated textile 5, 35, 40. The antimicrobial bioceramic coated textile construct 5, 35, 40 may comprise a blended textile 10, a first coating layer 25, and a second coating layer 30. The blended textile 10 comprises a first yarn 15 that forms a sheath around a second yarn 20 that forms the core. The first yarn 15 that forms the sheath includes a first yarn material. The first yarn 15 includes a first weave structure or configuration and the second yarn 15 includes a second weave structure or configuration. The core that forms the second yarn 20 comprises a second yarn material. The second yarn 20 may further comprise a monofilament or a multifilament yarn.

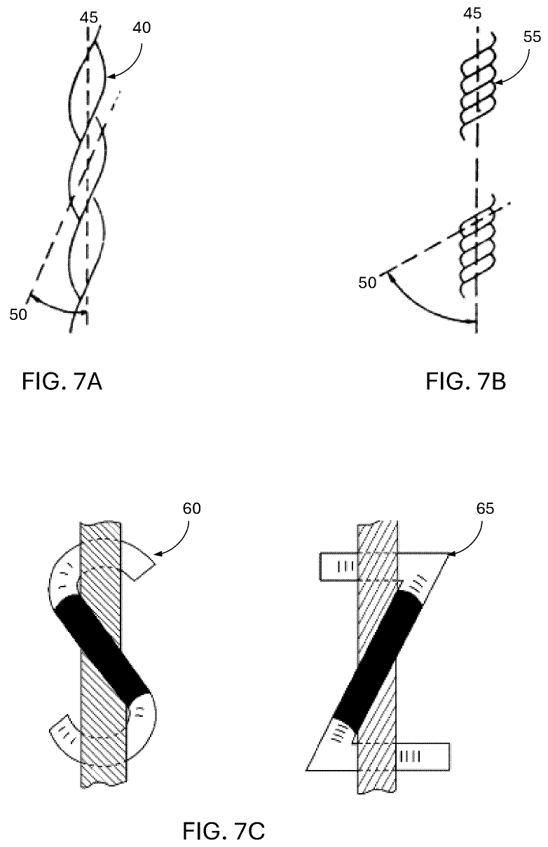

The blended textile 10 may comprise a first yarn 15 having a longitudinal axis 45, a first material, a first twist level 40, 55, a first twist angle 50, and a first twist direction 60, 65, and a second yarn 20 having a longitudinal axis 45, a second material, a second twist level 40, 45, a second twist angle 50 and a second twist direction 60, 65 as shown in FIGS. 7A-7C. The first yarn 15 comprises a first plurality of materials, first plurality of fibers, a longitudinal axis 45, a first plurality of twist levels 40, 55, a first plurality of twist directions 60, 65 and a first plurality twist angles 50. The second yarn 20 comprises a second plurality of materials, second plurality of fibers, a longitudinal axis 45, a second plurality of twist levels 40, 55, a second plurality of twist angles 50 and a second plurality of twist directions 60, 65.

In one embodiment, the first twist level 40, 55 of the first yarn 15 may comprise a different twist level than the second twist level 40, 55 of the second yarn 20. The first twist level 40, 55 of the first yarn 15 may comprise a same twist level than the second twist level 40, 55 of the second yarn 20. The first material of the first yarn 15 may comprise a different material than the second material of the second yarn 20. The first material of the first yarn 15 may comprise a same material than the second material of the second yarn 20. The first twist angle 50 of the first yarn 15 may comprise a different twist angle than the second twist angle 50 of the second yarn 20. The first twist angle 50 of the first yarn 15 may comprise a same twist angle than the second twist angle 50 of the second yarn 20. The first twist direction 60, 65 of the first yarn 15 may comprise a different twist direction than the second twist direction 60, 65 of the second yarn 20. The first twist direction 60, 65 of the first yarn 15 may comprise a same twist direction than the second twist direction 60, 65 of the second yarn 20.

In another embodiment, the first twist level 40, 55 of the first plurality of fibers may comprise a different twist level than the second twist level 40, 55 of the second plurality of fibers. The first twist level 40, 55 of the first plurality of fibers may comprise a same twist level than the second twist level 40, 55 of the second plurality of fibers. The first material of the first plurality of fibers may comprise a different material than the second material of the second plurality of fibers. The first material of the first plurality of fibers may comprise a same material than the second material of the second plurality of fibers. The first twist angle 50 of the first plurality of fibers may comprise a different twist angle than the second twist angle 50 of the second plurality of fibers. The first twist angle 50 of the first plurality of fibers may comprise a same twist angle than the second twist angle 50 of the second plurality of fibers. The first twist direction 60, 65 of the first plurality of fibers may comprise a different twist direction than the second twist direction 60, 65 of the second plurality of fibers. The first twist direction 60, 65 of the first plurality of fibers may comprise a same twist direction than the second twist direction 60, 65 of the second plurality of fibers.

In another embodiment, the first yarn 15 and/or the second yarn 20 comprises a single yarn. A single yarn is obtained by twisting a strand comprising a plurality of straight filaments into a twist level 40, 55, a twist angle 40, 55, and/or a twist direction 60, 65. Alternatively, the first yarn 15 and/or the second yarn 20 comprises a fiber-twisted single yarn. A fiber-twisted single yarn is obtained by twisting at least one or more of the plurality of fibers to a twist level 40, 55, a twist angle 50 and a twist direction 60, 65, then plying the twisted one or more plurality of fibers to create a single strand. The fiber-twisted single yarn may be optionally twisted to a twist level 40, 55, a twist angle 50 and a twist direction 60, 65 that is the same or different than the twists of the plurality of fibers.

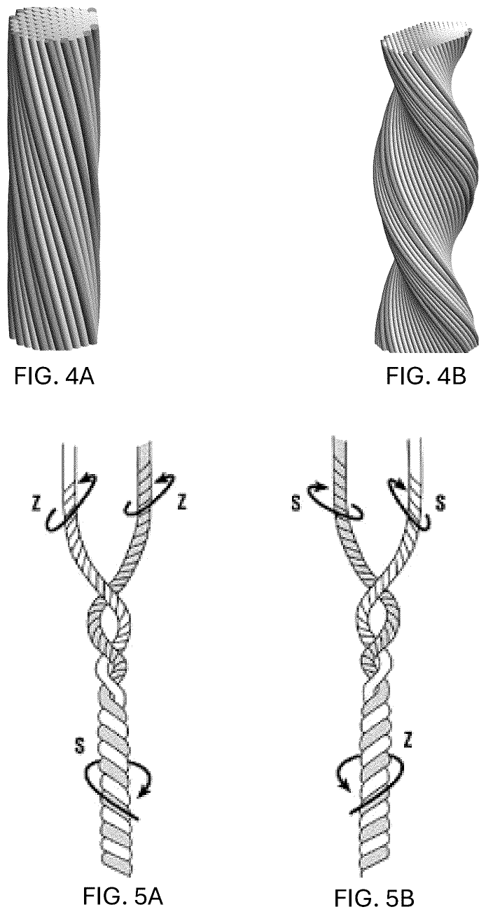

FIGS. 4A-4B depict an embodiment of a first yarn 15 and/or second yarn 20 illustrating a single yarn with different twist levels 40, 55. In one embodiment, the first yarn 15 comprises a first plurality of plied, straight fibers that are twisted to a first twist level 40, 55, a first twist direction 60, 65 and a first twist angle 50. The second yarn 20 comprises a second plurality of plied, straight fibers that are twisted to a twist level 40, 55, a twist direction 60, 65 and a twist angle 50. The first yarn material is the same or different than the second yarn material. The first plurality of fiber materials is the same or different than the second plurality of fiber materials.

FIGS. SA-5B depict alternate embodiment of a first yarn 15 and/or a second yarn 20 that is illustrating a fiber-twisted single yarn with different twist directions 60, 65. In one embodiment, the first yarn 15 comprising a first plurality of fibers and the second yarn 20 comprising a second plurality of fibers. Each of the first plurality of fibers including a first plurality of fiber materials, a first plurality of fiber twist levels 40, 55, a first plurality of fiber twist angle 50, and a first plurality of fiber twist direction 60, 65. Each of the second plurality of fibers including a second plurality of fiber materials, a second plurality of fiber twist levels 40, 55, a second plurality of fiber twist angles 50 and a second plurality of fiber twist directions 60, 65. Each of the twisted first plurality of fibers are plied to form first yarn 15 and each of the second plurality of twisted fibers are plied to form a second yarn 20. The first yarn 15 may be optionally further twisted or untwisted and the second yarn 20 may be optionally twisted or untwisted. The first yarn 15 and/or second yarn 20 twisting may be the same or different than the first plurality of fiber twisting and/or the second plurality of fiber twisting.

The twist level may comprise a twist-per-inch (TPI), a twist angle, a twist direction. The twist level may comprise a zero twist, a low twist, a moderate twist and a high twist. The low twist may comprise 1 to 20 TPI; the low twist may comprise 1 to 10 TPI; the low twist may comprise 1 to 5 TPI; the low twist may comprise 20 TPI or less; the low twist may comprise 15 TPI or less; the low twist may comprise 10 TPI or less; and the low twist may comprise 5 TPI or less. The moderate or medium twist may comprise 21 TPI to 60 TPI and the high twist may comprise 80 TPI or greater. The twist direction may comprise an S twist, Z twist direction and/or a combination thereof. The twist angle may comprise 10 degrees or greater; the twist angle may comprise 15 degrees or greater; the twist angle may comprise 20 degrees or greater; the twist angle may comprise 25 degrees or greater; the twist angle may comprise 30 degrees or greater; the twist angle may comprise a twist angle of 10 degrees to 75 degrees; and/or the twist angle may comprise a range of 10 degrees to 60 degrees.

The blended or composite textile 5, 30, 35 comprises a weave construction or configuration. The first yarn 15 and the second yarn 20 may be interlaced, interweaved or interconnected to create a blended or composite textile. The blended or composite textile may comprise a woven, a braided, a knitted textile construction, and/or any combination thereof. In one exemplary embodiment, the antimicrobial blended textile comprises a braided textile construction. The braided textile construction may comprise at least 8 spools or greater; at least 10 spools or greater; at least 12 spools or greater; at least 14 spools or greater; at least 16 spools or greater; and/or at least 17 spools or greater. Furthermore, the twisted, braided textiles are perceptibly rough and surface irregularities can be readily detected. The result of this rough and textured surface is to enhance the adherence of the bioceramic materials to the outer surface of the blended textile and/or the first coating layer. Similarly, the twisting of yarns and/or fibers help create a more compact and intertwined structure, making it more effective at retaining the applied or deposited ingredients.

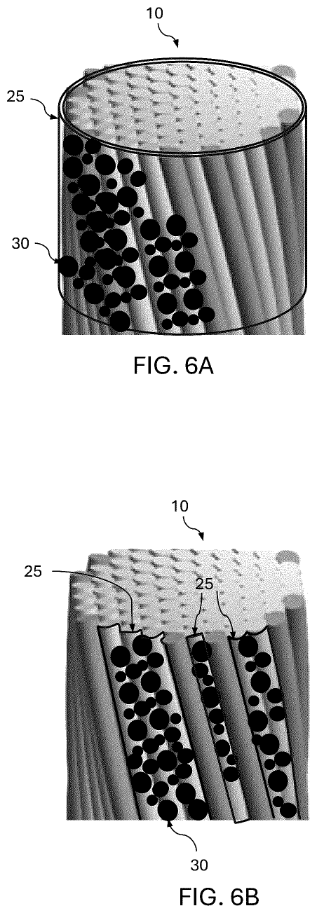

The blended or composite textile 5, 30, 35 comprises a first coating layer 25. The first coating layer 25 comprises a non-biodegradable polymer, the first coating layer 25 may be deposited or adhered a portion of the blended textile (as shown in FIGS. 1-3). The first coating layer 25 may be deposited or adhered to an outer surface of the blended textile 5, 30, 35. Alternatively, the first coating layer 25 may be deposited or adhered to a portion of the first yarn 15 and/or the second yarn 20 of the blended textile 5, 30, 35 as shown in FIGS. 6A-6B.

The first coating or first coating layer 25 may comprise a binder coating. The binder coating may comprise a binder coating dispersion or solution that includes finely divided polymer particles in a non-solvent. The binder coating or binder coating dispersion may further include an emulsifier or surfactant that is biocompatible. Preferably the non-solvent is an aqueous mixture or water. The person skilled in the art will be able to select suitable non-solvents and dispersion aids for a given coating polymer, or to select a commercially available dispersion that is suitable for use in present method based on present disclosures and his general knowledge.

In another embodiment, the binder coating solution or dispersion may comprise finely divided polymer particles in a solvent. The solvent may allow the polymer particles to be substantially or homogeneously dissolved. The person skilled in the art will be able to select a suitable solvent for a given binder coating based on his general knowledge, optionally supported by some experiments and/or literature.

The solvent may comprise a tetrahydrofuran (THF), methyltetrahydrofuran (m-THF), dimethylformamide (DMF), dimethylacetamide (DMAc), dimethylsulfoxide (DMSO), dioxane, dioxolane, or mixtures thereof. Suitable non-solvents for use in the treating solvent include for example lower aliphatic alcohols like ethanol, aliphatic esters, aliphatic ethers, and lower alkanes and alkenes. As indicated above, the non-solvent can preferentially evaporate from a mixture forming the treating solvent during the method.

In another embodiment, the binder coating or binder coating dispersion may comprise a solvent and a non-solvent. The solvent and non-solvent may comprise miscible solvents. It was observed that a good solvent for the polymer may, in addition to swelling a surface layer, also solubilize the layer; which may result in partial removal of the binder coating, or in ceramic particles being completely enclosed or embedded by binder coating. It has been surprisingly found that varying the composition of such treating solvent mixture, provides the skilled person with a tool to influence the degree of embedding of the ceramic particles in the layer of binder coating on the textile.

The finely divided polymer particles in a solvent or non-solvent comprises a concentration. The concentration of polymer may be chosen dependent on solubility and desired coating layer thickness. Generally, the concentration will be in the range 0.1-10 mass % of polymer particles in solvent. The solution contains e.g., at least 0.2, 0.5 or 1 mass %, and at most 8, 6, 4, 3 or 2 mass % of polymer particles.

The binder coating may be disposed onto a textile in different ways. The binder coating may be disposed onto an outer surface or outer diameter of the textile. The binder coating may be partially embedded within the textile. The binder coating may be fully embedded within the textile. Partially embedded means that the binder coating extends from the outer surface towards or through a portion of the wall thickness of the textile. Fully embedded means that the binder coating extends from the outer surface towards or through the wall thickness of the textile. The binder coating may be disposed onto the textile by dip coating or spray coating. After applying the solution or dispersion of the binder coating, the non-solvent or solvent is substantially removed by evaporation, if desired at elevated temperature to shorten time; to result in a layer of polymer particles on the textile. Furthermore, the solvent or non-solvent does not need to be completely removed at this stage, but a non-sticking surface layer is preferred to prevent treated fibers substantially adhering to each other.

The binder coating may comprise a binder coating thickness. In one embodiment, the thickness of the layer of the binder coating may be about half the size of the particles (taken as their d50 value, see hereinafter) of a bioceramic coating; so that the partially embedded particles still can protrude from the layer. In an embodiment of the method, the step of coating the textile having a layer of binder coating of at least 0.05 μm thickness. In further embodiments, the binder coating layer thickness is at least 0.01, 0.05, 0.1, 0.2, 0.3, 0.4, 0.6, 0.8, or 1 μm; whereas the layer thickness generally does not need to be more than 50, 40, 40, 20, 10, 5, or 2 μm. A relatively thin coating layer will have little effect on properties like flexibility of the textile.

The binder coating may further comprise a percent weight or mass increase (% w or % weight, % m or % mass) over the textile after the coating process. The thickness of the layer of the binder coating that is applied or the amount of binder coating applied may also be defined by the relative mass or weight increase of the coated product or coated textile after the coating process. The mass or weight increase upon coating the textile with binder coating is at least about 0.1, 0.2, 0.3, 0.4, or 0.5% mass, and/or at least 3, 2.5, or 2% mass.

The binder coating may further comprise a binder material. The binder material may comprise a ceramic, a polymer or a metal. The polymer may comprise a non-biodegradable polymer, a biodegradable polymer. The polymer may comprise a thermoset or thermoplastic polymer. The polymer may comprise a homopolymer, a copolymer and/or a block copolymer. The binder material may further include synthetic, semi-synthetic polymers. Semi-synthetic or bio-derived biocompatible polymers include materials like derivates of proteins and polysaccharides, such as cellulose. Synthetic biocompatible polymers include materials like poly (meth) acrylates, polyolefins, vinyl polymers, fluoropolymers, polyesters, polyamides, polysulfones, polyacrylics, polyacetals, polyimides, polycarbonates, polyethylenes, polyurethanes, including copolymers, compounds and blends thereof. Such synthetic polymers may be based on natural compounds like amino acids and/or on synthetic monomers.

In one exemplary embodiment, the binder material may comprise ultra-high molecular weight polyethylene (UHMWPE), lower density polyethylenes (LDPE), polyamide 66, polyurethanes; and/or polyethylene terephthalate (PET) with copolyesters. In other embodiments, the binder coating comprises a thermoplastic block copolymer. Block copolymers (or segmented) copolymers are polymers comprising blocks (also called segments) of polymers (including oligomers) that are chemically distinct, and that typically show different thermal and mechanical properties, and different solubilities. Often the blocks in a block copolymer comprising two (or more) types of blocks are referred to as being ‘hard’ and ‘soft’ polymer blocks, such different blocks resulting in microphase separation. The hard block in a block copolymer typically comprises a rigid or high modulus semi-crystalline or amorphous polymer, with—respectively—a melting temperature (Tm) or a glass transition temperature (Tg) higher than the use temperature, e.g. about 35° C.

The soft block in the block copolymer often comprises a flexible, amorphous polymer with a Tg lower than 35° C., preferably lower than 0° C. Thermal parameters like Tm and Tg are generally determined on dry samples; using well-known techniques like DSC or DMA. In such phase-separated block copolymers, the hard segments function as physical crosslinks for the flexible soft segments, resulting in materials having properties that may range from fairly stiff to flexible and elastic, depending on the ratio of hard to soft segments. When such block copolymer is heated above the softening point of the hard blocks, it will become a viscous fluid and may be processed into an article of desired shape and will solidify upon cooling. Such thermoplastic block copolymers showing flexibility or elastomeric character are generally referred to as thermoplastic elastomers, or TPEs.

In another embodiment, the binder material comprises a TPE material. The TPE comprises hard and/or soft blocks. The hard block comprises a polymer chosen from the group consisting of polyesters, polyamides, polystyrenes, polyacrylates, polyurethanes, polyolefins and/or any combination thereof. The soft block comprises a polymer chosen from the group consisting of polyethers, polyesters, polyacrylates, polyolefins and polysiloxanes, polyurethanes, and/or any combination thereof. Such polymers for the blocks are understood herein to include oligomers, homopolymers and copolymers, and polyesters are considered to include polycarbonates. Examples of TPE block copolymers are copolyester esters, copolyether esters, and copolycarbonate esters, wherein the hard blocks typically are based on semi-aromatic polyesters like polybutylene terephthalate (PBT); copolyester amides and copolyether amides; ethylene-propylene block copolymers; styrene-ethylene-butadiene block copolymers (SEBS); styrene-isobutylene block copolymers (SIBS); and polyurethanes comprising hard blocks based on diisocyanates and chain extenders, and polyester, polyether or polysiloxane soft blocks.

In another embodiment, the binder material may comprise a TPE material, the TPE material comprises a polyurethane or a polyurethane block copolymer. The polyurethane TPE (also referred to as TPU) comprises as soft block an aliphatic polyester dial, an aliphatic polyether dial, or a polysiloxane dial. The hard blocks of a block copolymer for use in the method of the invention, including polyurethane TPE, may have a molar mass of about 160 to 10,000 Da, and more preferably about 200 to 2,000 Da. The molar mass of the soft segments may be typically about 200 to 100,000 Da, and preferably about 400 to 9000 Da. The ratio of soft to hard blocks can be chosen to result in certain stiffness or hardness of the polymer. Typically, durometer hardness of the polyurethane as measured with the Shore test using A or D scales (e.g., ShA or ShD), may be from 40 ShA, or at least 50 or 60 ShA and up to 80 or 75 ShD, generally representing a flexural modulus range of about 10 to 2000 MPa.

In another embodiment, the binder material comprises a TPE material, the TPE material comprises a polyurethane TPE, the polyurethane TPE comprises an aliphatic polyether or an aliphatic polyester as soft block. More specifically, the polyurethane TPE comprises an aliphatic polycarbonate. Suitable polyethers include poly (propylene oxide) dials, poly (tetramethylene oxide) dials, and their copolymers. Suitable aliphatic polyesters are generally made from at least one aliphatic dicarboxylic acid and at least one aliphatic dial, which components are preferably chosen such that an essentially amorphous oligomer or polymer is formed having a Tg below 10, 0, or −10° C. Aliphatic polycarbonate dials are based on similar aliphatic dials as used for other polyester dials, and can be synthesized via different routes as known in the art, such as a poly (hexamethylene carbonate) dial. Commercially available examples of such polymers include the Bionate® PCU products (DSM Biomedical BV).

In another embodiment, the binder coating may comprise a first binder material and a second binder material. In one embodiment, the first binder material is different than the second binder material. In another embodiment, the first binder material is the same as the second binder material. Also, the binder coating may further comprise at least one additive. The at least one additive can be a targeted use of the coated product. The at least one additive may comprise antioxidants, processing aids, lubricants, surfactants, antistatic agents, colorants, radiopaque agents, fillers and/or any combination thereof. The at least one additive may be present in the typically effective amounts as known in the art, such as 0.01-5 mass % based on the amount of the polymer, preferably 0.01-1 mass %. In another embodiment, the binder coating does not include at least one additive.

The antimicrobial coated textile product 5, 35, 40 comprises a second coating layer 30. The second coating layer 30 comprises one or more bioceramic materials. The second coating layer 30 may be deposited or adhered to a portion of the first coating layer 25 (as shown in FIGS. 1-3). Alternatively, the second coating layer 30 may be deposited or adhered to a portion of the first coating layer 25 deposited or adhered to the first yarn 15 and/or the second yarn 15.

The second coating layer or second coating 30 may comprise one or more bioceramic coating materials or a bioactive ceramic coating materials. The second coating layer 30 comprises a dispersion including one or more bioactive ceramic particles and a solvent. Alternatively, the second coating layer may comprise a dispersion of one or more bioceramic particles and a non-solvent. The bioactive ceramic particles comprise all inorganic materials that show the capability of direct bonding to living bone, for example by formation of biologically active bone-like apatite through chemical reaction of the particle surface with surrounding body fluid. The bioactive ceramic particles may also include calcium phosphates, bioactive glass or bioglass or blends. Calcium phosphates may comprise dicalcium phosphate anhydrate (CaHPO4; or DCPA), dicalcium phosphate dihydrate (CaHPO4·2H2 O; DCPD), octacalcium phosphate (Ca8(HPO4)2·5H2O; or OCP), tricalcium phosphate (Ca3(PO4)2; or TCP), and hydroxyapatite (Ca10 (PO4MOH)2; or HA).

The bioactive glass blends may comprise at least two bioactive ceramic particle materials. In one embodiment, the blend may comprise HA and TCP. The blends may also comprise a bioactive ceramic particle and a small or trace amounts of other (inorganic) elements or ions, like Na, Mg, Fe, Zn, Ti, Ag, Cu or SO4, or CO 3 which may improve specific properties of the bioactive ceramic particles. In one embodiment, the bioceramic coating may comprise bioactive glass or BioGlass®. Bioglass® refers to mixed organic oxides that have a surface-reactive glass film compatible with tissues; and may be used as a surface coating in some types of medical and dental implants. The Bioglass® may comprise Bioglass® 45S5 grade, which is indicated to be a glass composed of 45 mass % Silicon Dioxide (SiO2), 24.5 mass % Calcium Oxide (CaO), 24.5 mass % Sodium Oxide (Na2O), and 6.0 mass % Phosphorous Pentoxide (P2O). The high ratio of calcium to phosphorus in this material would promote formation of apatite crystals; calcium and silica ions can act as crystallization nuclei. Glasses are non-crystalline amorphous solids that are commonly composed of silica-based materials with minor of other inorganic elements.

The bioactive ceramic particles may comprise a particle size. The particle size comprises a range of 0.01-10 μm. In another embodiment, the bioactive ceramic particle size may comprise a size smaller than 10 μm. In other embodiments, the ceramic particles size includes size of at least 20, 30, 50, 100, 200, 300, 400, or 500 μm. In other embodiments, the ceramic particle size may comprise at least 20 nm or greater. Further embodiments, the ceramic particles size comprises a size of at least 8, 7, 6, 5, 4, 3, 2 μm, or at most 1 μm. The particle size and particle size distribution can be measured with SEM or optical microscopy, or with (laser) light diffraction techniques. For example, a d50 value was measured with light diffraction according to ISO 13320:2009, e.g with a Malvern Mastersizer 2000 to help define the particle size of the bioceramic particles. Changing the particle size from μm to nm sizes and/or larger to smaller may increase surface area, allowing the release of more alkaline species, and consequently may display an enhanced microbial effect.

The bioceramic coating may comprise a percent mass (% mass) of bioactive ceramic particles within a treating solvent. The treating solvent may comprise a solvent or non-solvent. The % mass of bioactive ceramic particles in a treating solvent comprises 1-25% mass. In another embodiment, the % mass of bioactive ceramic particles in treating solvent or non-solvent comprises at least 22, 20, 18, 16, 14, 12 or 10% mass. In another embodiment, the % mass of bioactive ceramic particles in treating solvent or non-solvent comprises at least 1, 1.5, 2, 2.5, 3, or 5% mass of bioactive ceramic particles.

In another embodiment, the bioceramic coating may comprise a first one or more bioactive ceramic particles, a second one or more bioactive ceramic particles and a treating solvent. The treating solvent may comprise a standard solvent or a non-solvent. The treating solvent may comprise a solvent and a non-solvent. Alternatively, the bioceramic coating may comprise a first one or more bioactive ceramic particles and a second one or more bioactive ceramic particles within the dispersion. The first one or more bioactive ceramic particles may be different or the same as the second one or more bioactive ceramic particles. Additionally, the first bioactive ceramic particles may comprise a first bioactive ceramic particle size and the second bioactive ceramic particles may comprise a second bioactive particle size. The first bioactive ceramic particle size may be the same or different than the second bioactive ceramic particle size. The first one or more bioactive ceramic particles may comprise Bioglass® and a second one or more bioactive ceramic particles may comprise HAp.

The solvent or non-solvent used within the bioceramic coating may comprise a composition that interacts with the binder coating. The interaction of the solvent or non-solvent with the binder coating may include short contacting times to allow the solvent to effectively modify the surface of the binder coating, but evaporate or removed prior to implantation of the coated product. Modification of the surface of the binder coating may include swelling of a surface layer of the binder coating, tackify the surface layer of the binder coating, and/or solubilize the surface layer of the binder coating. The solubilization may result in partial removal of the binder coating to allow the bioceramic coating to be deposited on the binder coating or to be embedded or partially embedded into the binder coating or textile. The swelling of the surface layer may allow the bioactive ceramic particles sink or partially sink into a portion of the binder coating layer and/or textile. This may allow the bioceramic coating to be deposited on the binder coating surface, or to be embedded into a portion of the binder coating or fully embedded into a portion of the binder coating. In another embodiment, the solvent the bioceramic coating may comprise one or more bioceramic particles and a non-solvent.

The solvent may comprise a tetrahydrofuran (THF), methyltetrahydrofuran (m-THF), dimethylformamide (DMF), dimethylacetamide (DMAc), dimethylsulfoxide (DMSO), dioxane, dioxolane, or mixtures thereof. Suitable non-solvents for use in the treating solvent include for example lower aliphatic alcohols like ethanol, aliphatic esters, aliphatic ethers, and lower alkanes and alkenes. As indicated above, the non-solvent can preferentially evaporate from a mixture forming the treating solvent during the method.

In another embodiment, the bioceramic coating or the bioceramic coating dispersion may comprise a solvent and a non-solvent (e.g., or a treating solvent). The solvent and non-solvent may comprise miscible solvents. It was observed that a good solvent for the polymer may, in addition to swelling a surface layer, also solubilize the layer; which may result in partial removal of the binder coating, or in ceramic particles being completely enclosed or embedded by binder coating. It has been surprisingly found that varying the composition of such treating solvent mixture, provides the skilled person with a tool to influence the degree of embedding of the ceramic particles in the layer of binder coating on the textile.

The solvent and/or a non-solvent for the coating or binder polymer may be present in a percent volume (% Vol) within the bioceramic coating or the bioceramic coating dispersion. In one embodiment, the % volume may comprise a range of 2% to 98% volume. In another embodiment, the % volume may comprise at least 90, 80, 70, 70, 60, 50, 40, 30, 20, 10, 5 or 2 vol %.

The deposition or depositing of the bioceramic coating and/or bioceramic coating dispersion may comprise dip coating and spray coating onto the binder coating. Such coating methods allow to apply a thin layer of the dispersion on the surface of a complex shaped article like a textile within short time, optionally using multiple coating steps with intermediate drying, and with controllable contact time of coating polymer and dispersion, before removing excess dispersion and/or removing at least part of the treating solvent, e.g. by drying/evaporating and/or by rinsing with a rinsing solvent. Treating can be suitable performed at ambient conditions, but for example the temperature may also be increased, e.g. to shorten contacting and subsequent drying times.

In one embodiment, the bioceramic coating is deposited onto the binder coating or first coating layer 25 by dip coating. The dip coating may comprise submerge time, the submerge time is the total time the coated product, coated textile or textile is submerged in the bioceramic coating or the bioceramic coating dispersion. The submerge time includes periods of 1-20 seconds. Alternatively, the bioceramic coating is deposited onto the binder coating may a plurality of coating steps. By applying multiple short dip coating steps, the surface coverage can be more controlled with ceramic particles, rather than aiming to obtain a certain coverage in one step. The plurality of dip coating steps may include at least 2, 3, 4, 5, 6, 7, 8, 9 or 10 dip coating steps, optionally using intermediate drying periods to remove at least part of the treating solvent. A drying period can vary from 1 to 10 min, depending on conditions and volatility of treating solvent (or solvents contained therein). Suitable temperatures for coating and drying are in the range 10 to 150° C., depending on the softening temperature of the fibers of the textile or the binder coating; and is typically about 40-60° C., optionally in combination with reduced pressure and/or inert gas, like nitrogen flow. In another embodiment, the bioceramic coating is deposited onto the binder coating by spray coating. Spray coating allows the application of a plurality of thin layers after each attempt with preferably intermediate drying, for similar reasons as mentioned above for dip coating.

The solvent or non-solvent may be removed or substantially removed by a variety of different techniques. These techniques include evaporation, drying or rinsing. Drying conditions are dependent on the volatility of components to be removed, and the skilled person can determine suitable conditions. Drying can be done at ambient conditions, but also at elevated temperatures, under reduced pressure and/or by applying an inert gas flow. Rinsing aims to completely remove residual solvent or non-solvent and possibly other unwanted compounds, to make an article that will comply with requirements for medical implants.

In another embodiment, the bioceramic coating may comprise a total increase of percent weight (% wt) or percent mass (% mass) after coating application. The amount of bioceramic particles deposited onto the binder coating may be defined by the relative mass increase of the coated textile or product. The % weight or % mass increase of the coated fibrous product with a binder coating after application of the bioceramic coating or bioceramic dispersion and after removing or substantially removing the treating solvent or non-solvent comprises at least a 0.1, 0.2, 0.3, 0.4, or 0.5% mass increase. In another embodiment, the % mass increase is at least 20, 17, 15, 12, 10, 7, 5, 4, 3, 2.5, or 2% mass increase.

In another embodiment, first coating layer 25 and/or a second coating layer 30 may further comprise at least one metal oxide. The at least one metal oxide comprises iron, manganese, zinc, cobalt, gold, silver, copper and/or any combination thereof. Metal ions can enhance the bioactive responses of bioactive glass. More specifically, it can enhance the antimicrobial effect. Metal ions contain broad spectrum activities and interact with many different microbial intracellular components, resulting in the disruption of vital cell functions and eventually cell death.

In another embodiment, the antimicrobial coated textile 5, 35, 40 comprises a first yarn 15 having a first material and a second yarn 20 having a second material. Accordingly, the first yarn 15 comprises a first plurality of fibers having a first plurality of fiber materials, and the second yarn 20 comprises a second plurality of fibers having a second plurality of fiber materials.

The first material, the second material, the first plurality of fiber materials, the second plurality of fiber materials may comprise a ceramic, polymer or metal. The polymer may further include a thermoset or thermoplastic polymer. The materials may include a non-biodegradable material or polymer. The non-biodegradable polymers may comprise polyolefins, polyketones, polyamides, and polyesters. Suitable polyolefins include polyethylenes and polypropylenes, especially such polymers of high molar mass like ultra-high weight molecular polyethylene (UHMWPE). Suitable polyamides include aliphatic, semi-aromatic and aromatic polyamides, like polyamide 6, polyamide 66 and their copolymers, and poly(phenylene terephthalamide). Suitable polyesters include aliphatic, semi-aromatic and aromatic polyesters, like poly (l-lactic acid) and its copolymers, polyethylene terephthalate (PET), polytrimethylene terephthalate (PTT), polyethylene naphthalate (PEN), polyethylene furanoate (PEF) and liquid crystalline aromatic copolyesters.

The materials may further include biodegradable material. The biodegradable material may comprise a natural or synthetic biodegradable material. Natural biodegradable polymers include chitosan, silk fibroin, fibrinogen, collagen and hyaluronic acid. Synthetic biodegradable polymers include poly(ε-caprolactone) (PCL), PLA, PGA, copolymer PLGA, polytrimethylene carbonate (PTMC), and/or poly(p-dioxanone) (PDO). These materials have been proven to be biocompatible and have a controlled degradation rate, and their degradation products in-vivo have no toxic effects on tissues.

The materials may further include synthetic, semi-synthetic polymers. Semi-synthetic or bio-derived biocompatible polymers include materials like derivates of proteins and polysaccharides, such as cellulose. Synthetic biocompatible polymers include materials like poly (meth) acrylates, polyolefins, vinyl polymers, fluoropolymers, polyesters, polyamides, polysulfones, polyacrylics, polyacetals, polyimides, polycarbonates, polyethylenes, polyurethanes, including copolymers, compounds and blends thereof. Such synthetic polymers may be based on natural compounds like amino acids and/or on synthetic monomers.

The first material may be the same or different than the second material. The first plurality of fiber material may be the same or different than the second plurality of fiber materials. Each of the first plurality of fiber materials may be the same or different than each of the second plurality of fiber materials. In one exemplary embodiment, the first material and/or the first plurality of fiber materials may comprise polyester or polyethylene terephthalate (PET) and the second material and/or the second plurality of fiber materials may comprise ultra-high weight molecular polyethylene (UHWMPE).

Accordingly, the first material and/or the first plurality of fiber materials may comprise between 50% to 95% of the blended textile 10; the first material and/or the first plurality of fiber materials may comprise 50% to 90% of the blended textile 10; the first material and/or the first plurality of fiber materials may comprise 50% to 85% of the blended textile 10. The first material and/or the first plurality of fiber materials may comprise at least 50% or greater of the blended textile 10. The first material and/or the first plurality of fiber materials may comprise greater than 50% of the blended textile 10. The second material and/or the second plurality of fiber materials may comprise a range of 50% or less of the blended textile 10 and/or a range of 5% to 50% of the blended textile 10.

### Methods

In one embodiment, the method of manufacturing an antimicrobial bioceramic coated textile 5, 35, 40 comprises the steps of: twisting a first yarn to a first twist direction, a first twist angle and a first twist level and a second yarn to a second twist direction, a second twist angle and a second twist level, the first yarn including a first material and the second yarn including a second material, the first material comprising a different material than the second material; interlacing the first twisted yarn with the second twisted yarn to form a blended textile having a blended weave construction; applying a first coating layer to a portion of the blended textile, the first coating layer comprising a non-biodegradable polymer; applying a second coating layer to a portion of the first coating layer, the second coating layer comprises one or more bioceramic materials. The blended textile comprises a suture or an anchor sleeve. The twist level comprises a low or moderate twist level. The non-biodegradable polymer comprises Bionate. The one or more bioceramic particles may comprise hydroxyapatite (HA), Bioglass and/or Calcium Phosphate (CaP). The first twisted yarn material comprises greater than 50% of the blended textile. The first yarn material comprises polyester or PET and the second material comprises UHWMPE. The applying a first coating layer 25 to a portion of the blended textile comprises applying the first coating layer 25 to a portion of the first yarn 15 or a portion of the second yarn 20. The applying a first coating layer to a portion of the blended textile comprises applying the first coating layer to a portion of the first yarn or a portion of the second yarn.

In another embodiment, the method of manufacturing an antimicrobial bioceramic coated textile 5, 35, 40 comprises the steps of: twisting each of a first plurality of fibers of a first yarn to a first twist direction, a first twist angle and a first twist level and each of the second plurality of fibers of a second yarn to a second twist direction, a second twist angle and a second twist level, the first plurality of fibers including a first plurality of fiber materials of the first yarn and the second plurality of fibers including a second plurality of fiber materials of the second yarn, the first plurality of fiber materials comprising a different material than the second plurality of fiber materials; plying the first twisted plurality of fibers together to form the first yarn and the second plurality of fibers together to form the second yarn; interlacing the first yarn with the second yarn to form a blended textile having a blended weave construction; applying a first coating layer to a portion of the blended textile, the first coating layer comprising a non-biodegradable polymer; applying a second coating layer to a portion of the first coating layer, the second coating layer comprises one or more bioceramic materials. The blended textile comprises a suture or an anchor sleeve. The twist level comprises a low or moderate twist level. The non-biodegradable polymer comprises Bionate. The one or more bioceramic particles may comprise hydroxyapatite (HA), Bioglass and/or Calcium Phosphate (CaP). The first twisted yarn comprises greater than 50% of the blended textile. The applying a first coating layer to a portion of the blended textile comprises applying the first coating layer to a portion of the first yarn or a portion of the second yarn. The method of manufacturing an antimicrobial bioceramic coated textile 5, 35, 40 may further comprise the step of twisting the first yarn having a first plurality of twisted fibers to a first twist level, a first twist angle and a first twist direction, and the twisting the second yarn having a second plurality of twisted fibers to a second twist level, a second twist angle and a second twist direction.

### Experiments

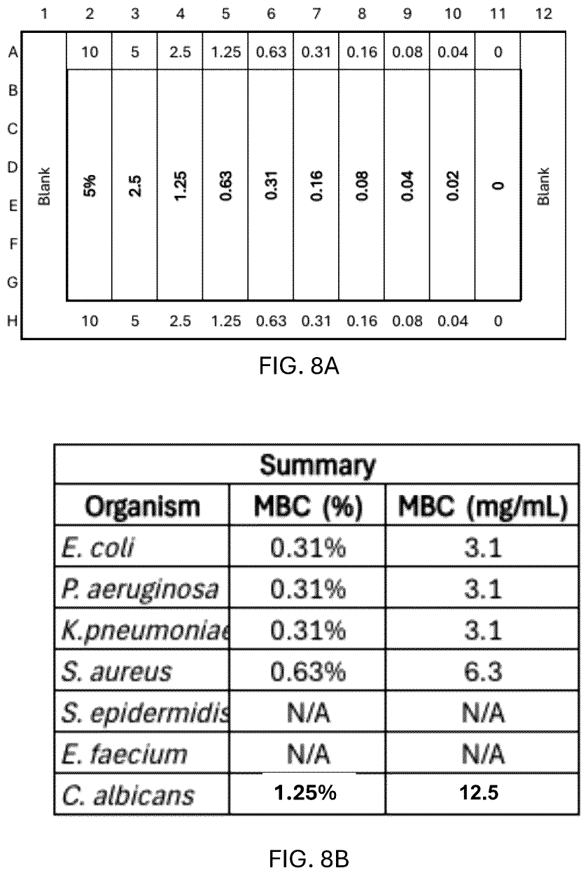

FIGS. 8A-8B, 9A-9C, and 10A-10B depict different experimental results from testing antimicrobial blended textile constructs to determine the minimum acceptable mass % of bioceramic required and the baseline antimicrobial properties of the samples to achieve an antimicrobial effect. The antimicrobial blended textile comprised a flat braid blended suture construction, the binding polymer and a bioceramic layer. The antimicrobial blended textile was manufactured by blending the fibers or yarns via (i) twisting them prior to braiding, and (ii) braiding them together in a 17 carrier braid machine. The flat braid blended textile comprises at least up to 75% PET to get enough surface area for the bioglass to adhere to create an antimicrobial or antibacterial layer, and the remaining of the second material. The second material comprised UHWMPE.

Once the braided textile construct was completed, the first binding layer was applied. After drying the first binding layer, the second bioceramic layer was applied using bioglass. Two types of antimicrobial blended textile constructs were provided, a concentration of 1 mass % and a 2.5 mass % was used for the bioceramic layer. Each of the antimicrobial blended textile constructs were severed into 1 cm testing samples.

### Determination of Minimum Bactericidal Concentration

The test was conducted to determine the minimum bactericidal concentration of the bioceramic was required to inhibit growth of various microbial species. The species that were tested were the Escherichia coli (strain 25922), Pseudomonas aeruginosa (strain 27312), Klebsiella pneumoniae (strain 4532), Staphylococcus aureus (strain 6538), Staphylococcus epidermidis (strain 14990), Enterococcus faecium (strain 51559) and Candida albicans (strain 10231).

The raw Bioactive glass was placed in 12-well culture plates designed for suspension culture experiments. The bioactive glass was prepared in SWF and diluted from 5% to 0.02% concentration as shown in FIG. 8A. The well culture plates were seeded with the different species and incubated at 37 degrees Celsius for 24 hours before imaging. The results in FIG. 8B indicate that a minimum of 1.25 mass % of raw bioactive glass would be required to inhibit growth of 5 of the 7 microbial species. No data was available for S. epidermidis because it did not survive in the testing conditions and E. faecium was not inhibited in SWF, therefore it maybe an improper medium or suspension or a minimum amount of Bioglass was necessary to inhibit growth of these two species.

### Determination of the Minimum Bioactive Calcium Component of Bioglass

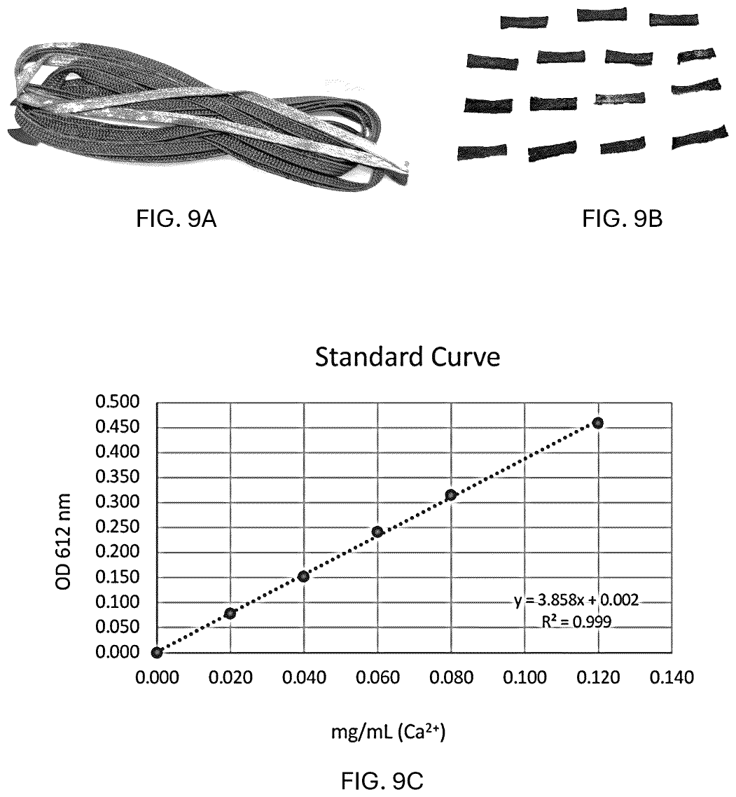

Calcium was determined to be the bioactive component in wound healing. The test was conducted to determine the minimum calcium required to quantify or provoke the bioactive response in a patient as shown in FIGS. 9A-9C. Only one of the two formulations were tested—the 2.5 mass % concentration was selected. The 2.5 mass % concentration samples were cut to 1 cm lengths. Each of the 1 cm samples were weighed and resulted in 4.7 mg/1 cm. Each sample was placed into 2 ml test tubes with 1 mm of 2 MHCL/0.2 M MES. The test tubes with the samples were vortexed and sonicated. Dilutions were performed and run against the calcium standard.

The data revealed that the calcium detected on the 1 cm suture samples was approximately 27.45 μg CaO. Bioglass is approximately 43-47 wt % SiO2, 22.5-26.5 wt % CaO, 24.5 wt % Na2O, and 6.0 wt % P20. Therefore 38.41 μg/ML of CaO is calculated approximately to be about 153.6 μg of Bioglass per 1 cm length. The data suggests that a minimum of at least 3 k μg Bioglass per 1 cm for an antimicrobial response.

### Determination of Antimicrobial Efficacy Via FDA Recognized Test

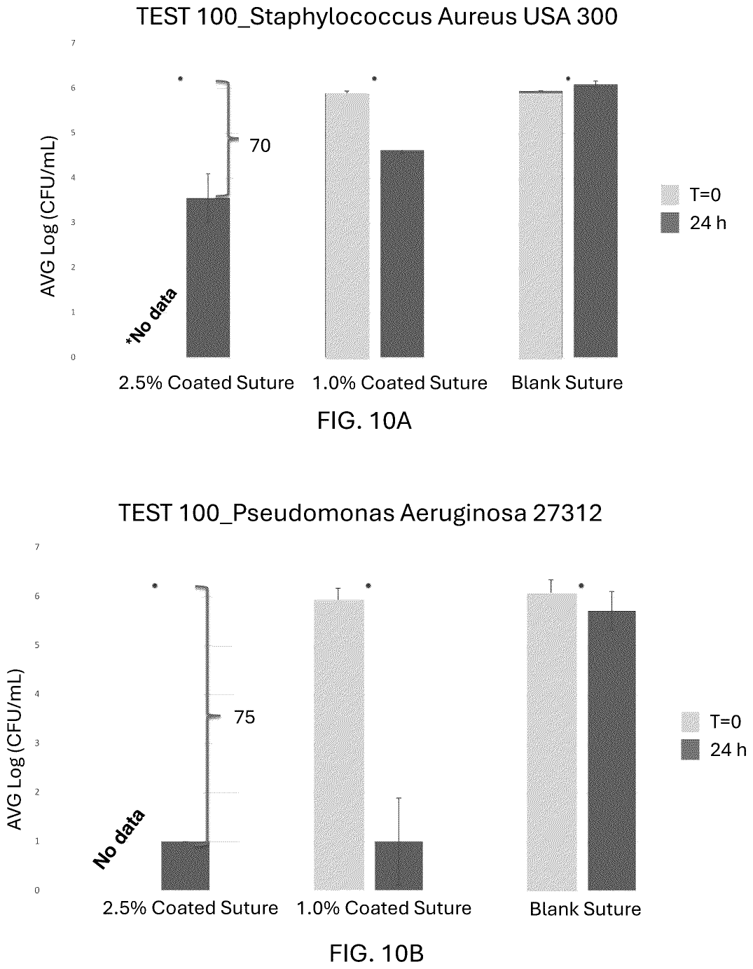

The test was conducted to determine the antimicrobial efficacy of the two formulations of the antimicrobial blended textile samples as shown in FIGS. 10A-10D. The test that was used is the AATCC Test Method 100. The two most common or aggressive microbial species were selected.—P. aeruginosa and S. aureus. The data results revealed a 3 log reduction 70 for the 2.5 mass % formulation of the S. aureus and a greater than 5 log reduction 75 of the 2.5 mass % formulation of the P. aeruginosa.

I/We claim:

1. An antimicrobial blended textile product comprises:
a textile, the textile comprising a first yarn having a first material, and a second yarn having a second material, the first yarn including a first plurality of fibers, a first yarn twist level, a first twist angle and a first twist direction and the second yarn including a second plurality of fibers, a second yarn twist level, a second twist angle, and a second twist direction, the first material is different than the second material;
a first coating, the first coating disposed onto a portion of the textile, the first coating comprising a first coating material and a first coating outer surface, the first coating material is a non-biodegradable polymer; and
a second coating, the second coating disposed onto a portion of the first coating, the second coating comprising a second coating material, the second coating material includes one or more bioceramic materials.

2. The antimicrobial blended textile of claim 1, wherein the textile comprises a braided textile.

3. The antimicrobial blended textile of claim 1, wherein the first yarn twist level is the same as the second yarn twist level.

4. The antimicrobial blended textile of claim 3, wherein the first yarn twist level and the second yarn twist level comprises a low twist level.

5. The antimicrobial blended textile of claim 4, wherein the low twist level comprises 20 twists-per-inch or less.

6. The antimicrobial blended textile of claim 1, wherein the first twist direction is the same or different as the second twist direction.

7. The antimicrobial blended textile of claim 1, wherein the one or more bioceramic materials comprises Bioglass.

8. The antimicrobial blended textile of claim 1, wherein the textile comprises a suture or an anchor sleeve.

9. The antimicrobial blended textile of claim 1, wherein the first yarn material comprises polyethylene terephthalate (PET) and the second yarn material comprises ultra-high weight molecular polyethylene (UHWMPE).

10. The antimicrobial blended textile of claim 1, wherein the first coating disposed onto a portion of the textile comprises the first coating disposed onto a portion of the first yarn of the textile or a portion of the second yarn of the textile.

11. The antimicrobial blended textile of claim 9, wherein the first yarn material comprises greater than 50% of the textile.

12. An antimicrobial blended textile comprises:
a textile, the textile comprising a first yarn having a first material, and a second yarn having a second material, the first yarn including a first plurality of fibers and the second yarn including a second plurality of fibers; the first plurality of fibers having a first fiber twist level and a first fiber twist direction and the second plurality of fibers having a second fiber twist level and a second fiber twist direction, the first material is different than the second material;
a first coating, the first coating disposed onto a portion of the textile, the first coating comprising a first coating material and a first coating outer surface, the first coating material comprises a non-biodegradable polymer; and
a second coating, the second coating disposed onto a portion of the first coating outer surface, the second coating comprising a second coating material and a second coating width, the second coating material includes one or more bioceramic materials.

13. The antimicrobial blended textile of claim 12, wherein the textile comprises a braided textile.

14. The antimicrobial blended textile of claim 12, wherein the first yarn twist level is the same as the second yarn twist level.

15. The antimicrobial blended textile of claim 14, wherein the first yarn twist level and the second yarn twist level comprises a low twist level.

16. The antimicrobial blended textile of claim 12, wherein the first twist direction is the same as the second twist direction.

17. The antimicrobial blended textile of claim 12, wherein the first coating material comprises Bionate and the one or more bioceramic materials comprises Bioglass.

18. The antimicrobial blended textile of claim 12, wherein the first yarn material comprises polyethylene terephthalate (PET) and the second yarn material comprises ultra-high weight molecular polyethylene (UHWMPE).

19. The antimicrobial blended textile of claim 12, wherein the first coating disposed onto a portion of the textile comprises the first coating disposed onto a portion of the first yarn of the textile or a portion of the second yarn of the textile.

20. The antimicrobial blended textile of claim 18, wherein the first yarn material comprises greater than 50% of the textile.
